(19) United States
(12) Patent Application Publication
(10) Pub. No.: US 2024/0257667 A1
(43) Pub. Date: Aug. 1, 2024

(54) 3D-PRINTED MEDICAL SIMULATOR AND METHOD

(71) Applicants: UNIVERSITY OF SOUTH FLORIDA, Tampa, FL (US); THE JOHNS HOPKINS UNIVERSITY, Baltimore, MD (US)

(72) Inventors: Stephen Alexander ROTTGERS, South St Petersburg, FL (US); Summer DECKER, Tampa, FL (US); Jonathan FORD, Tampa, FL (US)

(21) Appl. No.: 18/564,432

(22) PCT Filed: May 27, 2022
(86) PCT No.: PCT/US2022/031363

§ 371 Date: Nov. 27, 2023

Related U.S. Application Data
(60) Provisional application No. 63/194,700, filed on May 28, 2021.

(51) Int. Cl. G09B 23/30; A61B 34/10; B33Y 10/00; B33Y 50/00; B33Y 80/00

(52) U.S. Cl. CPC G09B 23/30; A61B 34/10; B33Y 10/00; B33Y 50/00; B33Y 80/00; A61B 2034/105

(57) ABSTRACT

A three-dimensional (3D) printed simulator that simulates at least a portion of a body is provided. The simulator includes a first simulated organ made of a first material having a first set of physical parameters, a second simulated organ made of a second material having a second set of physical parameters, and a transition layer made of a third material having a third set of physical parameters provided between the first simulated organ and the second simulated organ. The transition layer defines an anatomical plane between the first and second simulated organs.

### CROSS-REFERENCE TO RELATED APPLICATIONS

This application claims priority to U.S. provisional application Ser. No. 63/194,700, filed on May 28, 2021 and entitled “3D-Printed Medical Simulators,” which is incorporated herein by reference in its entirety.

### STATEMENT REGARDING FEDERALLY SPONSORED RESEARCH OR DEVELOPMENT

N/A

### BACKGROUND

Craniofacial surgeries in which osteotomies are performed adjacent to critical neurovascular structures represent a class of procedures within the field of plastic surgery having the highest risk of patient morbidity and mortality. These cases represent a small component of plastic surgery training and often residents' and fellows' first exposures to such procedures occur in the operating room in the later stages of their training. Accordingly, such individuals typically lack the procedural experience that is needed to become competent to perform procedures independently.

Healthcare simulation is an advancing field that has been shown to be a superior educational modality as compared to medicine's traditional apprenticeship model. In the typical scenario, a model or “simulator” of a particular portion of the anatomy is provided and a trainee can practice performing a surgical procedure on the simulator before attempting the procedure on a living patient. This form of training can result in increased surgical competence, decreased medical errors, and, ultimately, improved patient care outcomes.

Currently, there are no available craniofacial simulators that realistically represent the various tissues and structures associated with the cranium. While some craniofacial simulators have been developed, they are typically made from independently formed pieces of material that are assembled together with adhesive. Such simulators are not as useful as they could be given that the various tissues and structures, as well as their interrelationships, do not closely emulate those of a living patient. In addition, such simulators tend to be expensive as they normally require manual assembly by a human being.

In view of the above, it can be appreciated that it would be desirable to have a relatively low-cost, highly realistic craniofacial simulator that enables trainees to practice high-risk craniofacial surgical procedures before performing such procedures on a living patient.

### SUMMARY

In some embodiments, a three-dimensional (3D) printed simulator that simulates at least a portion of a body is provided. The simulator includes a first simulated organ made of a first material having a first set of physical parameters, a second simulated organ made of a second material having a second set of physical parameters, and a transition layer made of a third material having a third set of physical parameters provided between the first simulated organ and the second simulated organ. The transition layer defines an anatomical plane between the first and second simulated organs.

In some embodiments, a method for producing a simulator is provided. The method includes downloading files to a 3D printer that define the size, shape, configuration, position, and physical parameters of simulated organs that the simulator is to comprise. The method also includes downloading files to the 3D printer that define transition layers that are to be provided between the simulated organs, where the transition layers define anatomical planes between the simulated organs. The method further includes 3D printing the simulator using the 3D printer and the downloaded files in a continuous process in which printed layers are sequentially deposited one by one until the entire simulator and each of its simulated organs has been completed.

In some embodiments, a 3D printed simulator that simulates at least a portion of a body is providing, including a first simulated organ, a second simulated organ, and a transition layer. The first simulated organ is made of a first material and the second simulated organ is made of a second material. The transition layer is made of a third material provided between the first simulated organ and the second simulated organ, and defines an anatomical plane between the first and second simulated organs. The first simulated organ, the second simulated organ, and the transition layer are fabricated by a 3D printer en bloc.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a side perspective view of a craniofacial simulator, fabricated using a three-dimensional (3D) printer, according to some embodiments.

FIG. 2 is an isometric view of a 3D computer-aided design (CAD) model of the craniofacial simulator of FIG. 1.

FIG. 3 is a partial cross-sectional view of the 3D CAD model of FIG. 2.

FIG. 4 is a flow chart of a method for fabricating a simulator according to some embodiments.

FIG. 5 is a top side view of the craniofacial simulator of FIG. 1, illustrating performance of a simulated surgical procedure and, more particularly, dissection of different simulated organs (anatomical layers) of the simulator.

FIG. 6 is a top rear view of the craniofacial simulator of FIG. 1, illustrating performance of another simulated surgical procedure and, more particularly, dissection of different simulated organs (anatomical layers) of the simulator.

FIG. 7 is a top front view of the craniofacial simulator of FIG. 1, illustrating performance of a simulated surgical procedure and, more particularly, dissection of different simulated organs (anatomical layers) of the simulator.

FIG. 8 is another top rear view of the craniofacial simulator of FIG. 1, illustrating performance of another simulated surgical procedure and, more particularly, burr hole placement within the simulator.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

According to some embodiments, a relatively low-cost, highly realistic surgical simulator is provided for educational use as well as surgical use, for example, enabling trainees to practice surgical procedures, such as high-risk craniofacial surgical procedures. More specifically, three-dimensional (3D) printing can be used to create a variety of objects quickly at relatively low expense. This technology can be used to fabricate highly realistic surgical simulators that would enable trainees to practice surgical procedures, such as high-risk craniofacial surgical procedures, without putting actual patients at risk. High-fidelity craniofacial simulators would enable such trainees to experience the intricacies of the craniofacial anatomy and, therefore, could minimize damage to critical structures of an actual patient, which could lead to loss of vision, disfigurement, disability, and death.

Accordingly, in some embodiments, a 3D printer is used to produce, in a single continuous process, a complete craniofacial simulator that comprises every significant organ comprised by the human head and face. A simulator fabricated according to some embodiments not only simulates organs having physical properties that closely approximate those of the actual organs that they represent, but further closely emulates the interrelationships between those organs by using a transition material within transition layers between organs. As a result, the experience of practicing a surgical procedure on the simulator closely replicates the experience of performing the actual procedure on a living patient. Thus, with such a simulator, trainees can practice performing surgical approaches to the craniofacial skeleton, craniotomies, and facial osteotomies with complete autonomy. Also, as the simulators of some embodiments are 3D printed and, therefore, do not require manual assembly, they can be produced at lower cost than many existing simulators.

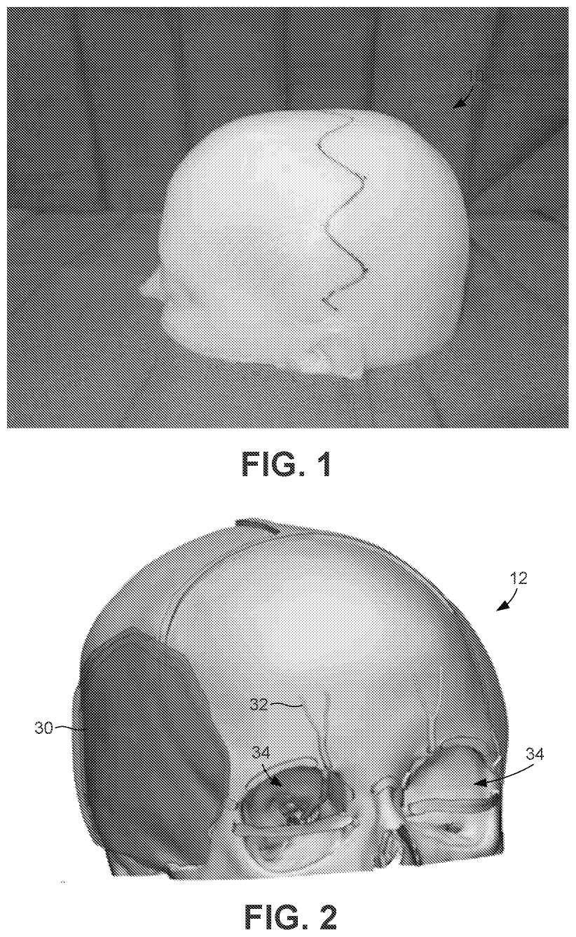

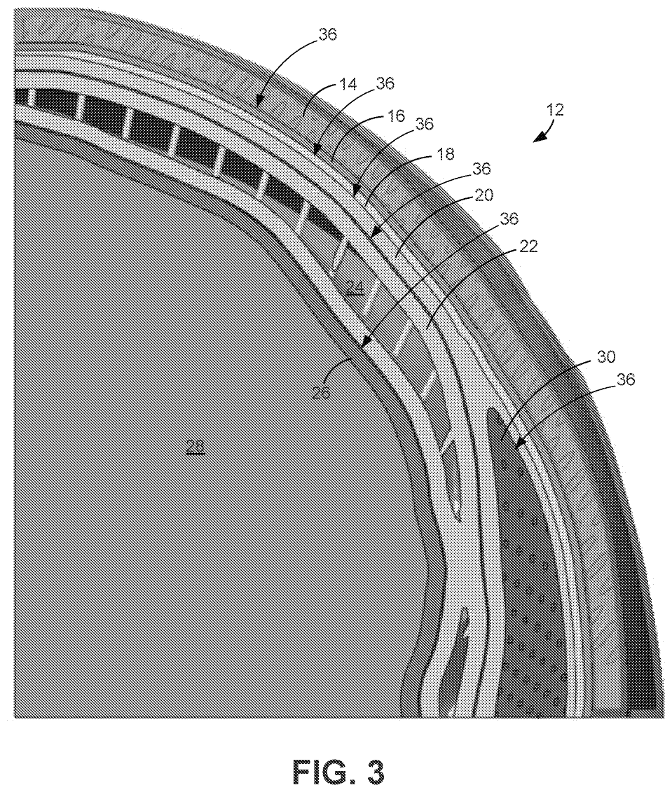

FIG. 1 illustrates a craniofacial simulator 10 according to some embodiments. The simulator 10 can be used as a surgical simulator (e.g., configured to enable a user to perform a simulated surgical procedure on the simulator 10 that simulates an actual surgical procedure that can be performed on a living patient) or as an educational tool (e.g., to educate users on the anatomy that the simulator 10 simulates). The simulator 10 can be fabricated using a 3D printer to include one or more, or all, of the relevant organs associated with the cranium and face, such as skin, fascia, muscles, periosteum, cranial bone, cranial sutures, zones of adherence, dura, orbital, and cranial contents. A 3D printer, such as the FDA-approved Stratasys J750 printer or Stratasys J735 printer, can be used to fabricate the simulator 10 based on a 3D computer-aided design (CAD) model 12, such as that shown in FIGS. 2 and 3. For example, the model 12 can be created based on computed tomography (CT) imaging data obtained from a living subject. In some embodiments, the model 12 can be created using segmented axial CT imaging data.

As shown in FIGS. 2 and 3, represented by the CAD model 12 are each of the primary organs (or tissues) of the head. With particular reference to FIG. 3, the model 12 includes skin 14, galea 16, innominate fascia 18, periosteum 20, the cranium 22 (including dipole space 24), dura 26, and the brain 28. Also, referring back to FIG. 2, the model 12 can further include certain muscles, such as the temporalis muscle 30, and nerves, such as the supraorbital nerve 32. Each of these organs can be modeled in terms of its size, shape, configuration, and position, as well as its physical parameters, such as density, hardness, flexibility, and elasticity. Furthermore, each of the parameters for each organ can be specified, for example, in STL files (or another file format used for 3D printing) that can be accessed by the 3D printer to fabricate the craniofacial simulator 10 based upon the model 12. That is, as multiple organs often reside in nearly each layer of material printed by the 3D printer, each printed layer can comprise materials having different parameters, based on content of the STL files, that emulate the characteristics of the actual tissues that are being represented. The 3D printer can then print the simulator 10 using an additive process in which multiple printed layers are sequentially deposited onto each other until the entire simulator 10 and each of its organs has been completed.

In some embodiments, different 3D printing materials may be selected for each tissue layer to best model its physical parameters as, for example, bone has a different hardness than soft tissue. In one specific example simulator 10 fabricated on a Stratasys J735 printer, VeroWhite can be used to simulate bone, Agilus 30 can be used for soft tissue layers, and SUP 706B support media can be used for intervening spaces (e.g., with varying thickness to modulate soft tissue adherence between layers). More specifically, with respect to the model 12 shown in FIG. 3, the skin layer 14 may comprise Agilus 30, the galea layer 16 comprises Agilus 30, the innominate fascia 18 may comprise SUP 706B, the periosteum 20 may comprise Agilus 30, the cranium 22 may comprise VeroWhite, the dura 26 may comprise Agilus 30, and the brain 28 may comprise SUP 706B. In other embodiments, different 3D printing materials (e.g., different polymers, resins, support media, etc.) may be used in some embodiments to best mimic the hard and soft tissue layers. For example, in addition to, or alternatively to, the above materials, TissueMatrix may be used for soft tissue layers, BoneMatrix may be used for bone, and GelMatrix may be used for support material, e.g., with a Stratasys J750 digital anatomy printer. Additionally, with respect to the view in FIG. 2, the cranium 22 and orbits 34 may be filled with support media, such as SUP 706B.

Thus, using anatomic segmentation software, the simulator 10 can be created by 3D printing hard and soft tissue structures en-bloc. Furthermore, by adjusting the CAD models 12, rapid development of simulators 10 with diverse pathology, anatomic variation, and patient-specific anatomy may be realized. For example, a patient-specific simulator may be created using a model 12 developed with patient-specific CT image data. As another example, disease-specific simulators 10 that comprise anatomy representing specific pathologies can be produced. Such simulators 10 can, for example, be fabricated based upon segmented axial CT imaging data of such pathologies. Furthermore, image data from more than one patient may be combined into a single model 12, e.g., to create a simulator 10 having multiple different pathologies.

Accordingly, in some embodiments, a library of CAD models, CT image data, or pre-segmented CT image data, may be developed directed to, for example, different patient sizes, demographics, pathologies, anatomic variations, etc. A user may then access the library to select a particular model 12 to rapidly fabricate with a 3D printer. 3D printing thus enables end users to fabricate their own simulators 10 using their own 3D printers by simply downloading models from the library and applying appropriate files, such as STL files. Additionally, in some embodiments, a user may also be able to customize or edit features of a particular model 12 prior to initial fabrication. Thus, in some embodiments, an entire line of disease-specific simulators 10 can be produced that includes simulators 10 each having an anatomy representing a specific craniofacial condition, thus enabling trainees to practice performing different procedures on various pathological conditions. Moreover, a patient-specific simulator 10 having the precise anatomy of a patient can be fabricated prior to surgery to enable a surgeon and/or trainee to practice a procedure before actually performing it on the patient, as many times as he or she wishes.

In light of the above, a simulator 10 can be created with different layers, each having different material properties that mimic the relative anatomical structure. A simulator 10, created en-bloc with these layers alone, may be beneficial for, e.g., patient education and counseling, surgical planning to conceptualize unique anatomy, producing customized implants, and creating cutting template guides. However, the inventors have determined that such a simulator 10 may not effectively mimic patient anatomy enough to be a useful tool for surgical training. More specifically, as shown the model 12 of FIG. 3, there are clear transition points, planes, or layers 36 between the tissue layers. When 3D printing a simulator, however, simply changing the material parameters (e.g., density) at those transition points 36 at which one simulated organ ends and another simulated organ begins within a printed layer can result in a lack of delineation between the organs (i.e., the anatomical or surgical planes between the organs). As a result, such a simulator does not properly mimic anatomy, including how it the organs respond to manipulation during a surgical intervention. That is, rather than exhibit varying degrees of adhesion between the layers, as naturally occurs in patient anatomy, all layers would be adhered together without discernible delineation between the layers.

To overcome these shortcomings, in some embodiments, a transition material that is different from the materials used to form adjacent simulated organs is provided between the organs to more clearly delineate the surgical planes 36 that separate them. With this addition, the simulator 10 can more accurately model normal anatomy and the experience of surgical dissection. More specifically, a transition material can be deposited at the transition planes 36 between each printed layer in which multiple organs are represented to establish the interfaces between those organs. Once the entire simulator 10 is 3D-printed, the transition material helps to more realistically represent the interfaces between adjacent organs. The transition layer 36 and accompanying transition material can thus simulate adhesion between the organs that would be encountered if the actual organs were dissected during surgical procedure.

Accordingly, some embodiments can provide, at a minimum, a 3D printed simulator 10 that simulates at least a portion of a body, including a first simulated organ made of a first material having a first set of physical parameters, a second simulated organ made of a second material having a second set of physical parameters, and a transition layer made of a transition material having a third set of physical parameters provided between the first simulated organ and the second simulated organ, where the transition layer defines an anatomical plane between the first and second simulated organs.

In some embodiments, the transition material can be a granular or gel material. Example transition materials can include, but are not limited to, SUP 706B and GelMatrix. For example, SUP 706B may be more granular while GelMatrix may be softer and more fluid. Other similar products may be used as transition materials in some embodiments (e.g., based on product availability and printer type). Additionally, in some embodiments, the transition material can have a hardness that is different than the hardnesses of the bone or soft-tissue layers of the simulator 10.

The nature of the transition material and, therefore, the transition layers 36 that result, can depend upon the organs that they separate as well upon the interface between the organs. That is, the type, properties, and/or thickness of the transition material can be varied throughout the transition layer 36 in order to properly mimic the real anatomical relationship between the adjacent tissues. As an example, the simulated periosteum 20 can have different levels of adhesion to the simulated cranium 22 depending upon the location on the cranium 22. For instance, the adhesion of the simulated periosteum 20 to the simulated cranium 22 can be greatest around the orbits of the eye formed by the simulated cranium 22, just as in the case of the actual anatomy. Thus, in some embodiments, the transition material may be thinner in those areas of greater adhesion along that layer 36.

Due to the provision of the transition layers 36 containing transition material, each of the anatomical layers of the craniofacial simulator 10 is well defined from its adjacent layers and can be dissected from those other layers in similar manner as can the actual tissues of a living patient. For example, the skin 14 can be dissected from the galea 16, which can be dissected from the innominate fascia 18, and so forth. Accordingly, in some embodiments, the transition layers 36 can be added as additional layers within the CAD model 12, and the properties (e.g., thickness) of transition material throughout each layer 36 be designated, including designated anatomic points of higher or lower adhesion throughout the individual layer 36. Additionally, while transition layers 36 are indicated in FIG. 3 between each organ, it should be noted that, in some embodiments, transition material may not be provided at each transition layer 36 and, as a result, those adjacent layers may be printed as one and adhered together.

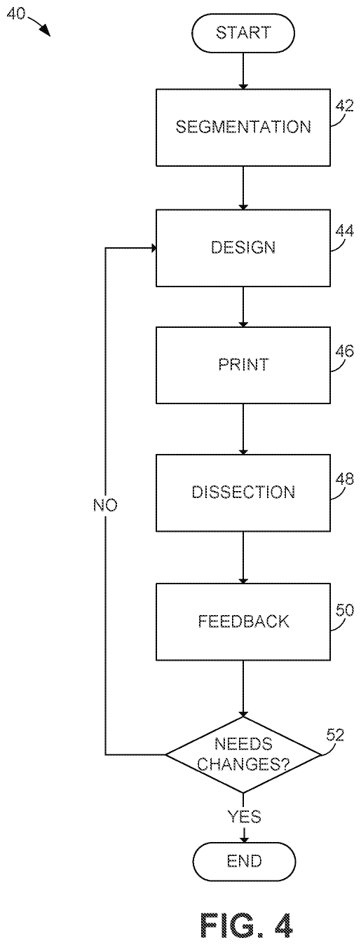

In view of the above, FIG. 4 illustrates a method 40 for fabricating a simulator 10 according to some embodiments. Generally, the method 40 can include an iterative workflow of a segmentation step 42, a design step 44, a printing step 46, a dissection step 48, and a feedback step 50. It should be noted that, in some embodiments, the method 40 may not include all steps 42-50 described herein, may include additional steps, or may include the steps in a different order than what is described.

In some embodiments, at the segmentation step 42, axial CT images can be segmented utilizing, for example, Mimics 23.0 (Materialise, Leuven, Belgium) to initially create 3D CAD models 12. Alternatively, in some embodiments, at the segmentation step 42, pre-segmented data may be downloaded from a library. At the design step 44, the model 12 can be refined to create the simulator 10. For example, in some embodiments, 3-Matic 15.0 (Materialise, Leuven, Belgium) can be utilized to vary tissue thicknesses and the spaces between planes to modify tissue pliability and design anatomic points of adhesion. Further, shore-A values can be varied to make tissues stiffer or more flexible. Transition layers can be created having varying thickness between organs, designed to control adherence between the anatomical layers and improve the haptic feedback during dissection at various locations throughout the model 12. In some embodiments, the model 12 may start as a small, multi-layered calvarial model 12 to define tissue layers and thicknesses, which is then further refined to create a full cranio-orbital model 12.

Completion of the design step 44 can result in files that define the size, shape, configuration, position, and physical parameters of simulated organs that the simulator 10 is to comprise, and files that define transition layers 36 that are to be provided between the simulated organs, defining anatomical planes between the simulated organs. In some embodiments, all layers may be defined in a single file.

At step 46, the model 12 can then be printed, e.g., using the materials described above, to create the simulator 10. For example, the file(s) can be downloaded to a 3D printer, which can then print the simulator 10. More specifically, in some embodiments, the 3D printer can be used to print the simulator 10 in an additive process, in which printer material is deposited printed layer by printed layer in a continuous process, thus printing all layers, including hard and soft tissue structures as well as transition layers, en-bloc. In other words, the simulator 10 is 3D printed in a continuous process in which printed layers are sequentially deposited one by one until the entire simulator 10 and each of its simulated organs has been completed.

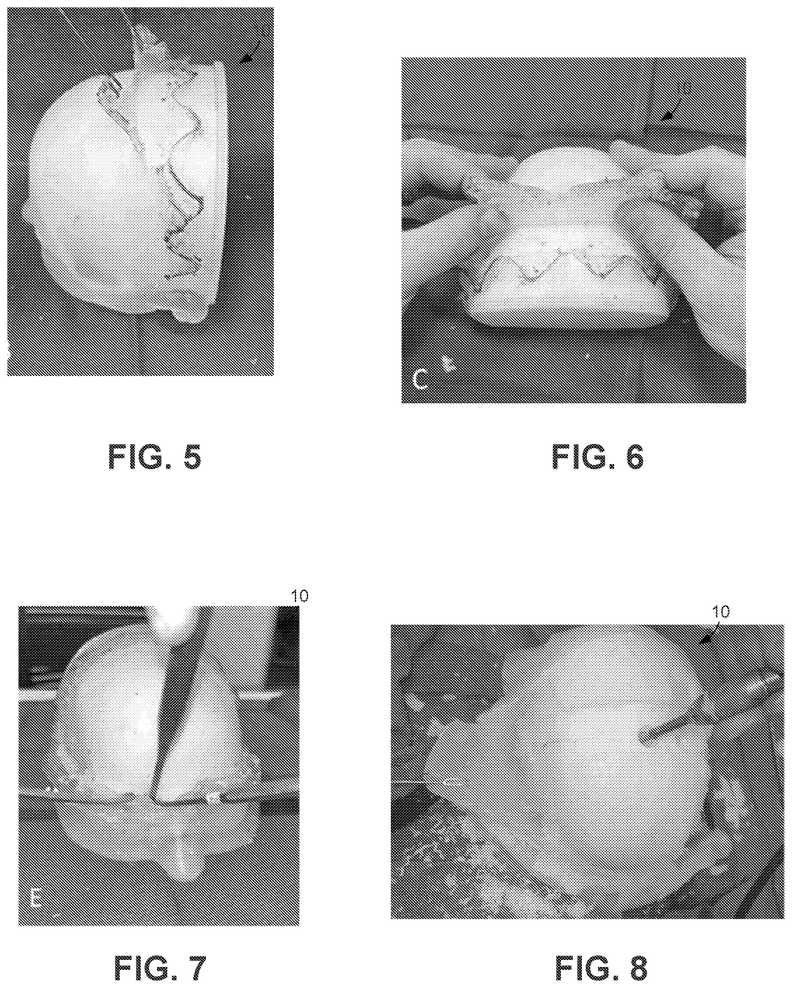

At step 48, the simulator 10 can be dissected and, at step 50 collaborative feedback may be obtained. For example, FIG. 5 illustrates a simulator 10 with a bicoronal incision made, with hooks used to retract the skin layer. As another example, FIG. 6 illustrates a simulator 10 with the bicoronal flap manually elevated. As yet another example, FIG. 7 illustrates a simulator 10 with an osteotomy of the supraorbital foramen performed. As yet another example, FIG. 8 illustrates a simulator 10 with burr holes placed. One or more of these procedures can be performed, for example, by an experienced surgeon, with respect to the feel of the tissue layers and their interactions during dissection. Feedback can then be given at step 50 that would allow the design to be changed to better mimic a patient's anatomy.

Following feedback step 50, if changes are desired, as determined at step 52, the method 40 may then return to the design step 44 to make integral modifications to the model 12 based on the feedback. If no feedback is desired, the model 12 may be set to be used for future simulators 10. Thus, after one or more intervals of design, printing, and improvement using the workflow method 40 described above, a cranioorbital model 12 can be created for anatomic design and the creation of multiple craniofacial simulators 10.

According to a study performed, a prototype craniofacial simulator 10 was fabricated using the above method and materials. It exhibited high anatomic fidelity and was successful as a platform for surgical simulation of a bicoronal incision, subgaleal and subperiosteal approach to the orbits, frontal craniotomy, and fronto-orbital osteotomy. High trainee satisfaction was also observed by those practicing on the simulator 10.

While the above disclosure is focused on the fabrication and use of craniofacial simulators 10 that simulate the organs of the head, it is noted that the concepts described herein can be extended to other simulators that represent other parts of the body (human or animal), either for use as surgical simulators or as educational tools as to the anatomy of the body. Accordingly, while craniofacial simulators have been described in detail, the present disclosure extends to any simulator that simulates either a part or the entirety of a body, whether it be human or animal. For example, the model design can be adjusted to design simulators for neurosurgical procedures such as external ventricular drain placement and hematoma evacuations. As another example, simulators of the hand anatomy can be created, having normal anatomy, or one or more different pathologies to be used as a training tool. As yet another example, an entire body simulator may be created, thus taking the place of a cadaver for teaching and/or surgical training purposes.

It is also noted that the concepts described above extend to all such simulators. For example, while the use of transition material has been discussed for delineating various organs/anatomical layers of the head, it is noted that such material can be used to delineate other simulated organs as well as to distinguish different tissues within the same simulated organ. For example, transition material can be used to define the various distinct segments of the liver, blood vessels or nerves that pass through an organ, fibers within simulated muscle, and so forth in different simulators.

The need for surgical simulation has grown in recent years while reduction in resident duty hours and a greater recognition of patient safety concerns have limited residents operative experience. This need is particularly accentuated in fields including procedures with low incidence of disease, translating into limited opportunities for trainees to learn (e.g., high acuity, low occurrence (HALO) procedures). Accordingly, some embodiments provide a simulator, and method of making a simulator, for medical and surgical education and training to meet this growing need. The simulators described herein are 3D printed en-bloc consisting of both bony and soft tissue, and transition material between organs to mimic real-life adhesion between organs. The utilization of 3D printing to fabricate these simulators reduces assembly time and removes manual labor of adhering together layers (as is required with other existing simulators). For example, other models previously produced either contain only bony tissue, which eliminates the opportunity to learn the handling of soft tissue, or are modular and require assembly, which is labor intensive. The nature of the 3D-printed simulator, printed en-bloc, can allow for easier production and ease of use in training programs. Furthermore, using pre-segmented image files to develop models can minimize costs and repetition of labor.

As used herein, unless otherwise defined or limited, the term “about” or “approximately” or “substantially” refers to variation in the numerical quantity that may occur, for example, through typical measuring and manufacturing procedures; through inadvertent error in these procedures; through differences in the manufacture, source, or purity of the ingredients used to make the compositions or mixtures or carry out the methods; and the like. Throughout the disclosure, the terms “about,” “approximately,” and “substantially” refer to a range of values ±20% of the numeric value that the term precedes.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. A three-dimensional (3D) printed simulator that simulates at least a portion of a body, the simulator comprising:
a first simulated organ made of a first material having a first set of physical parameters;
a second simulated organ made of a second material having a second set of physical parameters; and
a transition layer made of a third material having a third set of physical parameters provided between the first simulated organ and the second simulated organ, the transition layer defining an anatomical plane between the first and second simulated organs.

2. The 3D-printed simulator of claim 1, wherein the simulator is a surgical simulator configured to enable a user to perform a simulated surgical procedure on the simulator that simulates an actual surgical procedure that can be performed on a living patient.

3. The 3D-printed simulator of claim 1, wherein the simulator is an educational simulator configured to educate users on the anatomy that the simulator simulates.

4. The 3D-printed simulator of claim 1, wherein the first simulated organ comprises a simulated soft-tissue organ and the second simulated organ comprises simulated bone.

5. The 3D-printed simulator of claim 1, wherein the first simulated organ comprises a first simulated soft-tissue organ and the second simulated organ comprises a second simulated soft-tissue organ.

6. The 3D-printed simulator of claim 1, wherein the first simulated organ has a first hardness and the second simulated organ has a second hardness that is different from the first hardness.

7. The 3D-printed simulator of claim 6, wherein the transition layer has a third hardness that is different from the first and second harnesses.

8. The 3D-printed simulator of claim 1, wherein the transition layer simulates adhesion between the first and second simulated organs that would be encountered if the actual first and second organs were dissected during surgical procedure.

9. The 3D-printed simulator of claim 1, wherein the simulator is a craniofacial simulator that simulates the organs of the head.

10. The 3D-printed simulator of claim 9, wherein the craniofacial simulator includes simulated skin, galea, innominate fascia, periosteum, cranium, dura, and brain tissue.

11. The 3D-printed simulator of claim 10, wherein the craniofacial simulator is fabricated by a 3D printer via an additive process in which printer material is deposited printed layer by printed layer in a continuous process.

12. The 3D-printed simulator of claim 11, wherein multiple printed layers deposited by the 3D printer contain portions of multiple simulated organs and a portion of at least one transition layer.

13. A method for producing a simulator, the method comprising:
downloading files to a three-dimensional (3D) printer that define the size, shape, configuration, position, and physical parameters of simulated organs that the simulator is to comprise;
downloading files to the 3D printer that define transition layers that are to be provided between the simulated organs, the transition layers defining anatomical planes between the simulated organs; and
3D printing the simulator using the 3D printer and the downloaded files in a continuous process in which printed layers are sequentially deposited one by one until the entire simulator and each of its simulated organs has been completed.

14. The method of claim 13, wherein multiple printed layers deposited by the 3D printer comprise portions of multiple simulated organs and a portion of at least one transition layer.

15. The method of claim 13, wherein the transition layers simulate adhesion between adjacent simulated organs that would be encountered if the actual organs were dissected during surgical procedure.

16. A three-dimensional (3D) printed simulator that simulates at least a portion of a body, the simulator comprising:
a first simulated organ made of a first material;
a second simulated organ made of a second material; and
a transition layer made of a third material provided between the first simulated organ and the second simulated organ, the transition layer defining an anatomical plane between the first and second simulated organs,
the first simulated organ, the second simulated organ, and the transition layer fabricated by a 3D printer en bloc.

17. The 3D-printed simulator of claim 16, wherein the first simulated organ comprises a simulated soft-tissue organ and the second simulated organ comprises simulated bone.

18. The 3D-printed simulator of claim 16, wherein the first simulated organ, the second simulated organ, and the transition layer are fabricated by a 3D printer using a computer-aided design (CAD) model created with segmented axial computed tomography (CT) images.

19. The 3D-printed simulator of claim 16, wherein the third material includes varying thickness throughout the transition layer.

20. The 3D-printed simulator of claim 16, wherein the third material affects adhesion between the first simulated organ and the second simulated organ.
